(19) United States
(12) Patent Application Publication
(10) Pub. No.: US 2019/0160283 A1
(43) Pub. Date: May 30, 2019

(54) METHODS AND DEVICES FOR TREATING HPV-ASSOCIATED LESIONS USING NANOSECOND PULSED ELECTRIC FIELDS

(71) Applicant: Pulse Biosciences, Inc., Hayward, CA (US)

(72) Inventors: Richard Lee Nuccitelli, Millbrae, CA (US); Darrin Robert Uecker, San Mateo, CA (US)

(73) Assignee: Pulse Biosciences, Inc., Hayward, CA (US)

(21) Appl. No.: 15/824,967

(22) Filed: Nov. 28, 2017

(51) Int. Cl. A61N 1/36

(52) U.S. Cl. CPC A61N 1/36002; A61N 1/0408; A61N 1/36034

(57) ABSTRACT

In one aspect, methods of treating human papillomavirus (HPV)-associated growths are provided in which nano-pulse stimulation is applied at the site of a cancer. In another aspect, devices and computer systems for delivering nano-pulse stimulation for the treatment of HPV-associated growths are provided.

### CROSS-REFERENCES TO RELATED APPLICATIONS

NOT APPLICABLE

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### FIELD OF INVENTION

The present disclosure generally relates to methods and devices for treating tumors and lesions, such as human papillomavirus-associated tumors and lesions, using sub-microsecond, high electrical field pulses.

### BACKGROUND

Human papillomavirus (HPV) is a family of viruses for which more than 200 types have been identified (Brianti et al., New Microbiologica, 2017, 40:80-85). HPV infection is the most common sexually transmitted disease, and worldwide the risk of being infected at least once in a lifetime is about 50% (Handler et al., J Am Acad Dermatol., 2015, 73:743-756). Some types of HPV infection, such as HPV types 1, 2, 6, and 11, can cause abnormal growths that are benign, such as warts and sinonasal papillomas. Other types of HPV infections, such as types 16 and 18, are associated with cancerous and pre-cancerous growths.

HPV-associated anogenital and head and neck cancers cause significant morbidity and mortality worldwide. In general, HPV is detected in more than 90% of anal and cervical cancers, approximately 70% of oropharyngeal, vulvar, and vaginal cancers, and greater than 60% of penile cancers (Arbyn et al., Annals of Oncology: Official Journal of the European Society for Medical Oncology, 2011, 22:2675-2686). Cervical cancer is the number one cause of cancer-related death of women in developing countries (Soerjomataram et al., Lancet, 2012, 380:1840-1850). More specifically, HPV-induced cervical cancers are the third most common cancer in women and account for 7.5% of all female cancer deaths (zur Hausen et al., Nature Reviews Cancer, 2002, 2:342-350; Hung et al., Expert Opinion on Biological Therapy, 2008, 8:421-439).

Although effective prophylactic HPV vaccines aimed at targeting the L1 capsid protein have been developed and approved for use (Bosch et al., IBSCC Study Group: Journal of the National Cancer Institute, 1995, 87:796-802), uptake of these vaccines has been slow and they do not show therapeutic efficacy for individuals already infected with a high-risk HPV genotype or those harboring an HPV-transformed tumor (Reagen-Steiner et al., MMWR Morbidity and Mortality Weekly Report, 2015, 64:784-792). Because HPV-transformed cancers are expected to continue their upward trajectory in numbers in the foreseeable future, there remains a need for effective therapies that treat HPV-transformed cancers and other HPV-associated growths and lead to T cell immunity.

### SUMMARY

In one aspect, methods of treating a subject having a human papillomavirus (HPV)-associated growth are provided. In some embodiments, the method comprises applying a treatment of sub-microsecond pulses of an electric field with an intensity of 1 kV/cm or greater to the subject at a site of the HPV-associated growth, wherein the pulses are applied at a rate of energy deposition of up to about 14 watts/cc; thereby treating the HPV-associated growth.

In some embodiments, the HPV-associated growth is a benign growth. In some embodiments, the benign growth is a wart. In some embodiments, the benign growth is a sinonasal papilloma. In some embodiments, the benign growth is a laryngeal papilloma. In some embodiments, the HPV-associated growth is recurrent respiratory papillomatosis. In some embodiments, the HPV-associated growth is a pre-cancerous growth. In some embodiments, the HPV-associated growth is an HPV-associated cancer. In some embodiments, the HPV-associated cancer is a HPV-16 associated cancer or a HPV-18 associated cancer. In some embodiments, the HPV-associated cancer is cervical cancer, vulvar cancer, vaginal cancer, penile cancer, anal cancer, rectal cancer, or oropharyngeal cancer. In some embodiments, the pulses are applied at the rate of energy deposition configured to stimulate an immune response to the HPV-associated growth or metastasis.

In some embodiments, the treatment comprises applying the pulses at a rate of energy deposition of at least 0.1 watts/cc. In some embodiments, the treatment comprises applying the pulses at a rate of energy deposition that is between about 3 watts/cc and about 14 watts/cc. In some embodiments, the treatment comprises applying the pulses at a rate of up to 6 pulses per second.

In some embodiments, the pulses have a pulse amplitude of about 10 to 200 kV/cm. In some embodiments, the pulses have a pulse amplitude of about 30 kV/cm. In some embodiments, the pulses have a pulse duration of about 50 to 900 nanoseconds. In some embodiments, the pulses have a pulse duration of about 100 nanoseconds.

In another aspect, methods of inhibiting the recurrence of an HPV-associated growth or a metastasis of a cancer in a subject are provided. In some embodiments, the method comprises applying a treatment of sub-microsecond pulses of an electric field with an intensity of 1 kV/cm or greater to a site of the HPV-associated growth or metastasis in or on the subject, wherein the treatment comprises applying the pulses at a rate of energy deposition that is less than 14 watts/cc.

The pulses may be applied to stimulate an immune response to the HPV-associated growth or metastasis. In some embodiments, the treatment comprises applying the pulses at a rate of energy deposition that is less than 10 watts/cc. In some embodiments, the treatment comprises applying the pulses at a rate of energy deposition of about 3 watts/cc. In some embodiments, the treatment comprises applying the pulses at a rate of up to 5 pulses per second.

In some embodiments, the pulses have a pulse amplitude of about 10 to 200 kV/cm. In some embodiments, the pulses have a pulse amplitude of about 30 kV/cm. In some embodiments, the pulses have a pulse duration of about 50 to 900 nanoseconds. In some embodiments, the pulses have a pulse duration of about 100 nanoseconds.

In some embodiments, the treatment inhibits the recurrence of an HPV-associated growth. In some embodiments, the HPV-associated growth is a benign growth. In some embodiments, the HPV-associated growth is a pre-cancerous growth. In some embodiments, the HPV-associated growth is recurrent respiratory papillomatosis.

In some embodiments, the treatment inhibits the metastasis of a cancer. In some embodiments, the cancer is a virus-associated cancer. In some embodiments, the virus-associated cancer is an HPV-associated cancer. In some embodiments, the HPV-associated cancer is a HPV-16 associated cancer. In some embodiments, the HPV-associated cancer is cervical cancer, vulvar cancer, vaginal cancer, penile cancer, anal cancer, rectal cancer, or oropharyngeal cancer.

In yet another aspect, devices for delivering treatment of sub-microsecond pulses of an electric field are provided. In some embodiments, the device comprises:
a pulse generator configured to generate electric pulses;
an electrode assembly configured to deliver the electric pulses to a site of an abnormal growth in or on a subject, wherein the pulse generator and the electrode assembly are configured to apply a plurality of sub-microsecond pulses of an electric field with an intensity of 1 kV/cm or greater to the site of the abnormal growth; and
a processor operatively connected with a machine-readable non-transitory medium, the medium embodying information indicative of instructions for causing the processor to perform operations comprising:
   controlling the pulse generator to generate a number of pulses in a treatment session, each pulse having a pulse duration and a pulse amplitude calculated to deliver the pulses at a rate of energy deposition of up to 14 watts/cc.

In some embodiments, the operations comprise controlling the pulse generator to deliver the pulses at a rate of energy deposition that is less than 14 watts/cc. In some embodiments, the operations comprise controlling the pulse generator to deliver the pulses at a rate of energy deposition that is less than 10 watts/cc. In some embodiments, the operations comprise controlling the pulse generator to deliver the pulses at a rate of energy deposition that is at least 0.5 watts/cc. In some embodiments, the operations comprise controlling the pulse generator to deliver the pulses at a rate of energy deposition that is at least 0.1 watts/cc and less than 14 watts/cc (e.g., at least 0.5 watts/cc and less than 12 watts/cc, or less than 10 watts/cc). In some embodiments, the pulse generator is controlled to deliver pulses at a rate of energy deposition configured to stimulate an immune response to the abnormal growth (e.g., an HPV-associated growth or metastasis). The devices of the present disclosure may also comprise a user interface and they may be configured to allow a user to modify one or more parameters of pulses or other parameters of operation of the pulse generator.

In some embodiments, the operations comprise controlling the pulse generator to generate pulses having a pulse duration of about 50 to 900 nanoseconds. In some embodiments, the operations comprise controlling the pulse generator to generate pulses having a pulse duration of about 100 to 300 ns. In some embodiments, the operations comprise controlling the pulse generator to generate pulses having a pulse amplitude of about 10 to 200 kV/cm. In some embodiments, the operations comprise controlling the pulse generator to generate pulses having a pulse amplitude of about 20 to 35 kV/cm. A processor may be a computer processor, however, it is to be appreciated that the processor may be implemented by any combination of hardware, software, and firmware. Also, its functions as described herein may be performed in turn by any combination of hardware, software, and firmware.

In still another aspect, machine-readable non-transitory media are provided. In some embodiments, the medium embodies information indicative of instructions for causing a device comprising a pulse generator to perform operations for delivering treatment of sub-microsecond pulses comprising:
controlling the pulse generator to generate a number of pulses in a treatment session, each pulse having a pulse duration and a pulse amplitude calculated to deliver the pulses at a rate of energy deposition of up to 14 watts/cc to a site of an abnormal growth in or on a subject through an electrode assembly coupled to the pulse generator.

In some embodiments, the operations comprise controlling the pulse generator to deliver the pulses at a rate of energy deposition that is less than 14 watts/cc. In some embodiments, the operations comprise controlling the pulse generator to deliver the pulses at a rate of energy deposition that is less than 10 watts/cc. In some embodiments, the operations comprise controlling the pulse generator to deliver the pulses at a rate of energy deposition that is less than 12 watts/cc (e.g., less than 8 watts/cc). In some embodiments, the pulse generator is controlled to deliver pulses at a rate of energy deposition configured to stimulate an immune response to an HPV-associated growth or metastasis.

In some embodiments, the operations comprise controlling the pulse generator to generate pulses having a pulse duration of about 50 to 900 nanoseconds. In some embodiments, the operations comprise controlling the pulse generator to generate pulses having a pulse duration of about 100 ns. In some embodiments, the operations comprise controlling the pulse generator to generate pulses having a pulse amplitude of about 10 to 200 kV/cm. In some embodiments, the operations comprise controlling the pulse generator to generate pulses having a pulse amplitude of about 25-30 kV/cm.

In yet another aspect, computer systems for controlling a high voltage pulse generator are provided. In some embodiments, the system comprises:
a processor; and
a memory operatively coupled with the processor, the processor executing instructions from the memory comprising:
   program code for controlling a pulse generator to generate a number of pulses in a treatment session, each pulse having a pulse duration and a pulse amplitude calculated to deliver the pulses at a rate of energy deposition of up to 14 watts/cc to a site of an abnormal growth in or on a subject through an electrode assembly coupled to the pulse generator.

In some embodiments, the program code comprises controlling the pulse generator to deliver the pulses at a rate of energy deposition that is less than 14 watts/cc. In some embodiments, the operations comprise controlling the pulse generator to deliver the pulses at a rate of energy deposition that is less than 10 watts/cc. In some embodiments, the program code comprises controlling the pulse generator to deliver the pulses at a rate of energy deposition that is at least 0.1 watts/cc. In some embodiments, the program code comprises controlling the pulse generator to deliver the pulses at a rate of energy deposition that is at least 0.5 watts/cc and less than 14 watts/cc (e.g., at least 0.5 watts/cc and less than 12 watts/cc, or at least 1 watt/cc and less than 10 watts/cc). In some embodiments, the program code controls the pulse generator to deliver pulses at a rate of energy deposition configured to stimulate an immune response to the abnormal growth (e.g., an HPV-associated growth or metastasis).

In some embodiments, the program code comprises controlling the pulse generator to generate pulses having a pulse duration of about 50 to 900 nanoseconds. In some embodiments, the program code comprises controlling the pulse generator to generate pulses having a pulse duration of about 100 ns. In some embodiments, the program code comprises controlling the pulse generator to generate pulses having a pulse amplitude of about 10 to 200 kV/cm. In some embodiments, the program code comprises controlling the pulse generator to generate pulses having a pulse amplitude of about 10 to 30 kV/cm. Other features and advantages of the devices and methodology of the present disclosure will become apparent from the following detailed description of one or more implementations when read in view of the accompanying figures. Neither this summary nor the following detailed description purports to define the invention(s). The invention(s) is defined by the claims.

### BRIEF DESCRIPTION OF THE DRAWINGS

FIG. 1A illustrates a non-limiting example of a diagram of the ratio of the amounts of activated caspase 3/7 in treated to untreated cells at 3 hours post Nano-Pulse Stimulation (NPS) treatment for a range of NPS energy densities. FIGS. 1B-1D illustrate non-limiting examples of the distribution of treated C3.43 tumor cells categorized as being in early apoptosis, late apoptosis/necrotic, very late stage cell death, or live. FIG. 1B demonstrates an example of distribution of treated C3.43 tumor cells at 1 hour post-treatment with the indicated NPS energy density. FIG. 1C demonstrates an example of distribution of treated C3.43 tumor cells at 3 hours post-treatment with the indicated NPS energy density. FIG. 1D demonstrates an example of distribution of treated C3.43 tumor cells at 24 hours post-treatment with the indicated NPS energy density.

FIGS. 2A-2B illustrate non-limiting examples of T cells induced by NPS treatment in mice. FIG. 2A demonstrates an example of HPV16 E7 specific T cell response. FIG. 2B demonstrates an example of ampitope T cell response. FIG. 2C illustrates a non-limiting example of a diagram showing an inverse correlation between the percentage of tumor infiltrating lymphocytes vs size of the tumor in NPS-treated mice.

FIGS. 3A-3D illustrate non-limiting examples of growth curves of primary tumors (solid line) and re-challenge tumors (dotted lines) following NPS treatment. FIG. 3A demonstrates an example of a growth curve of primary and re-challenge tumors in mice treated with the indicated NPS conditions. FIG. 3B demonstrates an example of a growth curve of primary and re-challenge tumors in mice treated with the indicated NPS conditions. FIG. 3C demonstrates an example of a growth curve of primary and re-challenge tumors in mice treated with the indicated NPS conditions and selective depletion of CD4. FIG. 3D demonstrates an example of a growth curve of primary and re-challenge tumors in mice treated with the indicated NPS conditions and selective depletion of CD8.

FIGS. 4A-4B illustrate non-limiting examples of growth curves of tumors in NPS-treated mice. FIG. 4A demonstrates an example of a growth curve of re-challenge tumors in mice treated with NPS. FIG. 4B demonstrates an example of a growth curve of re-challenge tumors in mice treated with NPS with or without CD4- or CD8-depletion. FIGS. 4C-4D illustrate non-limiting examples of diagrams showing the survival of mice receiving NPS treatment. FIG. 4C demonstrates an example of a diagram showing the survival of mice treated with NPS. FIG. 4C demonstrates an example of a diagram showing the survival of mice treated with NPS with or without CD4- or CD8-depletion.

### DETAILED DESCRIPTION

### I. Introduction

Human papillomavirus (HPV) is a family of DNA viruses that is associated with the induction of abnormal growths in infected individuals. These HPV-associated growths can be benign, pre-cancerous, or cancerous. Without being bound to a particular theory, it is believed that different HPV types cause different types of growths. For example, certain types of HPV are considered “low-risk” or “non-oncogenic,” such as but not limited to HPV types 1, 2, 4, 6, and 11. These HPV types are typically associated with benign growths, such as genital warts (HPV-6 and HPV-11), common warts (HPV-2, HPV-27, and HPV-57), flat warts (HPV-3 and HPV-10), plantar and palmar warts (HPV-1), sinonasal papillomas (HPV-6 and HPV-11), and recurrent respiratory papillomatosis. (HPV-6 and HPV-11). See, Egawa et al., Virus Research, 2017, 231:119-127. Other types of HPV are considered “high-risk” types, such as but not limited to HPV types 16 and 18, and are associated with numerous types of cancers, including but not limited to cervical cancer, anal cancer, and oropharyngeal cancers. High-risk types of HPV are also associated with pre-cancerous lesions, such as intraepithelial lesions, that may subsequently develop into cancer.

Several conventional therapies, such as Loop electrosurgical excision, radiotherapy, Mohs surgery, and cryotherapy, have been used to ablate HPV-associated cancers. However, these therapies have varying degrees of efficacy. Furthermore, there is a significant level of disease recurrence associated with these ablative therapies. See, e.g., Juhl et al., International Journal of STD & AIDS, 2016, 27:1071-1078.

The present disclosure relates to the finding that the application of sub-microsecond, high electrical field pulses (also referred to herein as “nanosecond pulsed electric field” or “nano-pulse stimulation”) to a model of human papillomavirus (HPV)-transformed tumors is effective at eliminating primary tumors in a single treatment through the induction of immunogenic cell death. Thus, sub-microsecond electric pulses represents a novel therapy for ablating HPV-associated cancers as well as other HPV-associated growths.

The present disclosure also relates to the surprising discovery that the rate of energy deposition of the sub-microsecond electric pulses affects the initiation of an adaptive immune response in the subject that protects the subject from tumor recurrence. As described in Example 1 below, it has been surprisingly found that, for example, in a mouse HPV16 tumor model, application of nano-pulse stimulation at a rate of about 3 watts/cc resulted in tumor ablation and protected mice from tumor rechallenge. However, when nano-pulse stimulation was applied at a higher rate of about above 14 watts/cc, rechallenged tumor growth was not inhibited. Without being bound to a particular theory, it is believed that application of nano-pulse stimulation at a moderate rate induces an adaptive immune response to unknown tumor antigens. Thus, by applying nano-pulse stimulation at a moderate rate, tumor recurrence and metastasis can be inhibited or prevented. This adaptive immune response can also be induced to inhibit or prevent the recurrence of other abnormal HPV-associated growths, such as warts and lesions.

### II. Definitions

As used herein, the term “human papillomavirus” or “HPV” refers to a virus that is classified within the human papillomavirus group of DNA viruses. HPV types can be subdivided into “low-risk” and “high-risk” groups with respect to the frequency in which the HPV type is associated with a cancer. In some embodiments, the HPV is an HPV type that is a “high risk” type for association with a cancer, such as but not limited to HPV-16, HPV-18, HPV-31, HPV-33, HPV-34, HPV-35, HPV-39, HPV-45, HPV-51, HPV-52, HPV-56, HPV-58, HPV-59, and HPV-68. In some embodiments, the HPV is an HPV type that is a “low risk” type for association with a cancer, such as but not limited to HPV-1, HPV-2, HPV-3, HPV-4, HPV-6, HPV-7, HPV-10, HPV-11, HPV-27, HPV-54, HPV-57, HPV-60, HPV-63, and HPV-65.

As used herein, the term “HPV-associated growth” refers to an abnormal growth of cells that is caused by an HPV infection. An HPV-associated growth can be a benign growth, a pre-cancerous growth, or a cancerous growth.

The term “cancer” refers to a disease characterized by the uncontrolled growth of aberrant cells. The term includes all known cancers and neoplastic conditions, and cancers of all stages and grades including pre- and post-metastatic cancers. Examples of different types of cancer include, but are not limited to, digestive and gastrointestinal cancers such as gastric cancer (e.g., stomach cancer), colorectal cancer, gastrointestinal stromal tumors, gastrointestinal carcinoid tumors, colon cancer, rectal cancer, anal cancer, bile duct cancer, small intestine cancer, and esophageal cancer; breast cancer; lung cancer; gallbladder cancer; liver cancer; pancreatic cancer; appendix cancer; prostate cancer; penile cancer; cervical cancer; vaginal cancer; ovarian cancer; vulvar cancer; renal cancer; cancer of the central nervous system; skin cancer (e.g., melanoma); lymphomas; gliomas; choriocarcinomas; head and neck cancers; oropharyngeal cancers; osteogenic sarcomas; and blood cancers.

The term “tumor” refers to an abnormal growth of cells, e.g., on or within a subject. The tumor may be malignant or benign. In some embodiments, a tumor comprises one or more cancerous cells. In some embodiments, a tumor comprises one or more cancerous cells of an HPV-associated cancer.

The term “sub-microsecond pulses of an electric field” or “sub-microsecond pulsed electric field” refers to an electric pulse with a pulse width (duration) of less than 1000 nanoseconds, e.g., a pulse width of at least 0.1 nanoseconds and less than 1000 nanoseconds. Sub-microsecond pulses often have high peak voltages, such as 10 kilovolts per centimeter (kV/cm), 20 kV/cm, or higher. An example of the application of sub-microsecond pulsed electric fields to a tumor is disclosed in U.S. Pat. No. 9,512,334, the contents of which are incorporated by reference herein.

The terms “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In some embodiments, the subject is a human.

The terms “treating” and “treatment” are used herein to generally mean obtaining a desired physiologic and/or pharmacologic effect, and may refer to any indicia of success in the treatment or amelioration of a condition, disorder, or disease (e.g., cancer), including any objective or subjective parameter such as abatement, remission, improvement in patient survival, increase in survival time or rate, diminishing of symptoms or making the condition, disorder, or disease more tolerable to the patient, slowing in the rate of degeneration or decline, or improving a patient's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

The terms “operatively connected,” “coupled,” or “attached,” as used herein, means directly or indirectly operatively connected, coupled, or attached through one or more intervening components.

### III. Methods of Treatment

In one aspect, methods of treating a subject having a human papillomavirus (HPV)-associated growth are provided. In some embodiments, the method comprises applying a treatment of sub-microsecond pulses of an electric field with an intensity of 1 kV/cm or greater to a site of the HPV-associated growth in the subject, wherein the pulses are applied at a rate of energy deposition of up to about 14 watts/cc; thereby treating the HPV-associated growth. The proposed methods may be substantially automated, which means that some or all of the steps could be performed automatically, for example, by a processor or other computing device. It does not exclude, however, that the user may intervene and participate, for example, by giving an alternative command through a user interface, or override the automated command.

In another aspect, methods of inhibiting the recurrence of an HPV-associated growth or a metastasis of a cancer (e.g., an HPV-associated cancer) in a subject are provided. In some embodiments, the method comprises applying a treatment of sub-microsecond pulses of an electric field with an intensity of 1 kV/cm or greater to a site of the HPV-associated growth or metastasis in the subject, wherein the treatment comprises applying the pulses at a rate of energy deposition that is less than 14 watts/cc; thereby inhibiting the recurrence of the HPV-associated growth or metastasis.

In some embodiments, the subject to be treated is a human. In some embodiments, the subject is a human adult (e.g., an adult from age 18 and up). In some embodiments, the subject is a human child (e.g., a child from age 12-17).

In some embodiments, the HPV-associated growth is a benign growth. In some embodiments, the HPV-associated growth is a pre-cancerous growth. In some embodiments, the HPV-associated growth is a cancerous growth.

### HPV-Associated Growths

In some embodiments, methods of treating or inhibiting the recurrence of a benign HPV-associated growth are provided. In some embodiments, the HPV-associated growth is a benign growth. In some embodiments, the benign growth is a benign lesion, wart, or papilloma. In some embodiments, the benign growth is a wart, e.g., a plantar wart, palmar wart, mosaic wart, common wart or verruca vulgaris, flat wart, nongenital wart, genital wart, or anogenital wart. In some embodiments, the benign growth is a lesion, e.g., a mucosal lesion or a cutaneous lesion. In some embodiments, the benign growth is a papilloma, e.g., a sinonasal papilloma or a laryngeal papilloma.

In some embodiments, the HPV-associated growth is a recurrent growth, e.g., a recurrent benign growth. In some embodiments, the HPV-associated growth is laryngeal papillomatosis, also referred to as recurrent respiratory papillomatosis (RRP), which is a disease characterized by recurrent growth of benign papillomas in the respiratory tract (e.g., in the larynx).

In some embodiments, the HPV-associated growth is a pre-cancerous growth. In some embodiments, the pre-cancerous growth is an intraepithelial lesion, such as a squamous intraepithelial lesion (SIL). In some embodiments, the SIL is a low-grade SIL. In some embodiments, the SIL is a high-grade SIL. In some embodiments, the intraepithelial lesion is a cervical intraepithelial neoplasia (CIN).

In some embodiments, the HPV-associated growth is a growth that is associated with a low-risk HPV type. For example, in some embodiments, the HPV-associated growth is a benign growth (e.g., a benign lesion, wart, or papilloma) that is associated with a low-risk HPV type. In some embodiments, the low-risk HPV type is HPV-1, HPV-2, HPV-3, HPV-4, HPV-6, HPV-7, HPV-10, HPV-11, HPV-27, HPV-54, HPV-57, HPV-60, HPV-63, or HPV-65. In some embodiments, the HPV-associated growth is a wart, lesion, or papilloma that is associated with HPV-1, HPV-2, HPV-3, HPV-4, HPV-6, HPV-7, HPV-10, HPV-11, HPV-57, or HPV-65.

In some embodiments, the HPV-associated growth is a pre-cancerous growth that is associated with a high-risk HPV type. For example, in some embodiments, the HPV-associated growth is a CIN or a high-grade SIL that is associated with a high risk type of HPV, such as but not limited to HPV-16, HPV-18, HPV-31, HPV-33, HPV-35, HPV-39, HPV-45, HPV-51, HPV-52, HPV-56, HPV-58, HPV-59, or HPV-68. In some embodiments, the HPV-associated growth is a CIN or a high-grade SIL that is associated with HPV-16, HPV-18, or HPV-31.

### HPV-Associated Cancers

In some embodiments, methods of treating an HPV-associated cancer or inhibiting the recurrence or metastasis of an HPV-associated cancer are provided. In some embodiments, the HPV-associated cancer is a cancer that is associated with a high risk type of HPV. In some embodiments, the high risk type of HPV is HPV-16, HPV-18, HPV-31, HPV-33, HPV-34, HPV-35, HPV-39, HPV-45, HPV-51, HPV-52, HPV-56, HPV-58, HPV-59, or HPV-68. In some embodiments, the HPV-associated cancer is a cancer that is associated with HPV-16 or HPV-18. In some embodiments, the HPV-associated cancer is a cancer that is associated with HPV-16.

In some embodiments, the HPV-associated cancer is cervical cancer, vulvar cancer, vaginal cancer, penile cancer, anal cancer, rectal cancer, or oropharyngeal cancer. In some embodiments, the HPV-associated cancer is cervical cancer. In some embodiments, the HPV-associated cancer is anal cancer. In some embodiments, the HPV-associated cancer is oropharyngeal cancer.

In some embodiments, the subject has been diagnosed as having an HPV-associated cancer (e.g., a cancer that is associated with a high risk type of HPV). Methods of detecting HPV are known in the art. In some embodiments, the method of detecting HPV comprises genotyping for the presence of a high-risk HPV type. As a non-limiting example, the Hybrid Capture 2 test (“hc2”) or the digene HC2 DNA test (Qiagen, Germantown, Md.) can be used to detect the presence of a high-risk HPV type.

HPV-associated cancers found in anogenital, oral mucosa, and other cutaneous sites are an increasing global health burden. Several conventional therapies have been used to ablate HPV-associated cancers, including Loop electrosurgical excision (Frega et al., European Review for Medical and Pharmacological Sciences, 2017, 21:2504-2511), radiotherapy (Langendijk et al., Recent Results in Cancer Research, 2017, 206:161-171), Mohs surgery (Marchionne et al., Anais brasileiros de dermatologia, 2017, 92:95-99), and cryotherapy (Juhl et al., International Journal of STD & AIDS, 2016, 27:1071-1078, Bertolotti et al., Journal of the American Academy of Dermatology, 2017, doi:10.1016/j.jaad.2017.04.012), however efficacies vary from 23% to 94% and there remains a significant level of recurrence (Juhl et al.). None of these ablative therapies to date have been shown to induce an immunogenic response against targeted tumors, which may be an underlying explanation for the rates of disease recurrence.

It has been reported that NPS treatment of non-viral-transformed tumor cells results in the release of the three classical danger-associated molecular pattern molecules (DAMPs): calreticulin, ATP, and HMGB1 (Nuccitelli et al., Journal for Immunotherapy of Cancer, 2017, 5:32-45), all associated with programmed cell death. NPS has been shown to generate transient nanopores in the plasma membrane and organelle membranes of treated tumor cells that allow the movement of ions across these membranes (see, e.g., Pakhomov et al., Biochem Biophys Res Commun, 2009, 285:181-185). An immediate consequence of this is an increase in cytoplasmic Ca²⁺ and the permeabilization of the endoplasmic reticulum (ER) and mitochondria (Vernier et al., Biochem Biophys Res Commun, 2003, 310:286-295; Vernier et al., Conf Proc IEEE Eng Med Biol Soc, 2011, 743:743-745). The spike in cytoplasmic Ca²⁺ has been found to stimulate reactive oxygen species generation (ROS) (Nuccitelli et al., Biochem Biophys Res Commun, 2013, 435:580-585) and to trigger apoptosis when a sufficient number of spikes are generated. Simultaneously, ER-permeabilization stresses this organelle, and combined with ROS can lead to the translocation of the ER protein, calreticulin, to the plasma membrane where it initiates an “eat me” signal to dendritic cells (Garg et al., EMO J., 2012, 31:1062-1079). Upon processing tumor proteins, the dendritic cells may then present tumor neoantigens to the immune system and generate specific CD8⁺ cytotoxic T cells that will circulate in the body to seek out tumor cells expressing these novel targets (Nuccitelli et al., PLoS One, 2015, 10(7):e0134364). Immunogenomic approaches have shown that successful immunotherapy against HPV16-driven tumors that has resulted in complete remission of metastatic events is primarily driven by T cells that recognize mutated neoantigens or cancer germline antigens (Stevanovic et al., Science, 2017, 356:200-205). This is the response that NPS treatment is believed to induce.

The current landscape of cancer immunotherapy relies on creating or driving forward immune responses to tumor associated antigens. With NPS therapy, as described in the present disclosure, not only are primary HPV-tumors in mice eliminated, but a CD8-dependent protection against HPV-tumor rechallenge events is also induced.

### Other Cancers

In some embodiments, methods of inhibiting the recurrence or metastasis of a cancer are provided. In some embodiments, the cancer is a cancer other than an HPV-associated cancer. In some embodiments, the cancer is a virus-associated cancer. In some embodiments, the cancer is not a virus-associated cancer.

In some embodiments, the cancer is a digestive or gastrointestinal cancer, such as gastric cancer (e.g., stomach cancer), colorectal cancer, gastrointestinal stromal tumors, gastrointestinal carcinoid tumors, colon cancer, rectal cancer, anal cancer, bile duct cancer, small intestine cancer, and esophageal cancer. In some embodiments, the cancer is breast cancer. In some embodiments, the cancer is lung cancer. In some embodiments, the cancer is gallbladder cancer. In some embodiments, the cancer is liver cancer. In some embodiments, the cancer is pancreatic cancer. In some embodiments, the cancer is a urogenital cancer, such as prostate cancer, penile cancer, testicular cancer, cervical cancer, vaginal cancer, ovarian cancer, vulvar cancer, renal cancer, or bladder cancer. In some embodiments, the cancer is a skin cancer (e.g., melanoma). In some embodiments, the cancer is a blood cancer, such as lymphoma, leukemia, or myeloma. In some embodiments, the cancer is a head, neck, or oropharyngeal cancer. In some embodiments, the cancer is a glioma. In some embodiments, the cancer is a bone cancer (e.g., osteogenic sarcomas).

### Parameters for Sub Microsecond Electric Pulses

The methods of the disclosure comprise applying sub-microsecond pulses of an electric field to a subject, e.g., at a site of an HPV-associated growth or at a tumor-associated site. Electrical pulse generators for delivering sub-microsecond high voltage pulses to biological cells and tissues are described in the art. See, e.g., US 2013/0150935, U.S. Pat. Nos. 8,512,334, and 9,101,764, each of which is incorporated by reference herein.

In some embodiments, one or more of the following parameters of the sub-microsecond pulses may be adjusted to provide optimal treatment: (1) rate of energy deposition (watts/cc); (2) pulse amplitude (kV/cm); (3) pulse duration (ns); (4) pulse number applied; and (5) pulse application rate (pulses per second). Various parameters may be adjusted automatically by the processor or by a user, who may intervene and participate, for example, by giving an alternative command through a user interface, or override the automated command. The parameters may be inputted through one or more input and/or output components for transmitting output to and/or receiving input from one or more other components (such as one or more displays, touch screens, keyboards, mice, track pads, track balls, styluses, pens, printers, speakers, cameras, video cameras, and so on). Without being bound to a particular theory, it has been surprisingly found that when an aberrant growth (e.g., an HPV-associated growth) is treated with sub-microsecond high voltage pulses, the initiation of an immune response at the site of the aberrant growth is dependent upon the rate of energy deposition of the pulses. Specifically, as disclosed herein in the Examples section below, it has been surprisingly found that there is a maximum rate of energy deposition above which an immune response and subsequent protection from recurrence of regrowth is not initiated.

For methods of treating a benign HPV-associated growth or a method of treating an HPV-associated cancer, in some embodiments, the method comprises applying a treatment of sub-microsecond pulses at a rate of energy deposition of up to about 14 watts/cc. In some embodiments, the pulses are applied at a rate of energy deposition that is from about 0.1 watts/cc to about 14 watts/cc, e.g., from about from 0.2 watts/cc to about 12 watts/cc, from about 0.5 watts/cc to about 10 watts/cc, from about 0.5 watts/cc to about 8 watts/cc, from about 0.5 watts/cc to about 6 watts/cc, from about 1 watt/cc to about 12 watts/cc, from about 1 watt/cc to about 10 watts/cc, from about 1 watt/cc to about 8 watts/cc, from about 2 watts/cc to about 12 watts/cc, from about 2 watts/cc to about 10 watts/cc, from about 2 watts/cc to about 8 watts/cc, from about 2 watts/cc to about 6 watts/cc, from about 3 watts/cc to about 12 watts/cc, from about 3 watts/cc to about 10 watts/cc, from about 3 watts/cc to about 8 watts/cc, or from about 3 watts/cc to about 6 watts/cc. In some embodiments, a method of treatment as disclosed herein (e.g., a method of treating a benign HPV-associated growth or a method of treating an HPV-associated cancer) comprises applying a treatment of sub-microsecond pulses at a rate of energy deposition of up to about 14 watts/cc, including for example, of about 0.1 watts/cc, about 0.2 watts/cc, about 0.3 watts/cc, about 0.4 watts/cc, about 0.5 watts/cc, about 1 watt/cc, about 1.5 watts/cc, about 2 watts/cc, about 2.5 watts/cc, about 3 watts/cc, about 4 watts/cc, about 5 watts/cc, about 6 watts/cc, about 7 watts/cc, about 8 watts/cc, about 9 watts/cc, about 10 watts/cc, about 11 watts/cc, about 12 watts/cc, or about 13 watts/cc.

For methods of inhibiting the recurrence of a growth (e.g., a tumor or lesion) or inhibiting the metastasis of a cancer, in some embodiments, the pulses are applied at a rate of energy deposition that is less than 14 watts/cc. In some embodiments, the pulses are applied at a rate of energy deposition that is less than 12 watts/cc, e.g., less than 11 watts/cc, less than 10 watts/cc, less than 9 watts/cc, less than 8 watts/cc, less than 7 watts/cc, or less than 6 watts/cc.

It will be recognized by a person of skill in the art that a constant rate of energy deposition can be maintained at a specific level, or a rate of energy deposition can be maintained above or below a specified level, while varying other parameters of the sub-microsecond pulses (e.g., pulse amplitude, pulse duration, pulse number, and pulse application rate). As a non-limiting example, in some embodiments, a specified rate of energy deposition can be maintained if the pulse amplitude is increased by decreasing another parameter, e.g., by decreasing pulse duration, pulse number, or pulse application rate.

In some embodiments, sub-microsecond pulses of an electric field are applied with an intensity of 1 kV/cm or greater, e.g., at least 5 kV/cm, 10 kV/cm, 15 kV/cm, 20 kV/cm, 25 kV/cm, 30 kV/cm, 35 kV/cm, 40 kV/cm, 45 kV/cm, 50 kV/cm, or greater, to an HPV-associated tumor site. In some embodiments, a pulse amplitude in the range of 1 kV/cm to 200 kV/cm, e.g., from 5 kV/cm to 200 kV/cm, from 5 kV/cm to 100 kV/cm, from 5 kV/cm to 50 kV/cm, from 10 kV/cm to 200 kV/cm, from 10 kV/cm to 150 kV/cm, from 10 kV/cm to 100 kV/cm, from 10 kV/cm to 50 kV/cm, from 10 kV/cm to 30 kV/cm, from 15 kV/cm to 45 kV/cm, from 15 kV/cm to 35 kV/cm, from 20 kV/cm to 200 kV/cm, from 20 kV/cm to 150 kV/cm, from about 20 kV/cm to 100 kV/cm, from 20 kV/cm to 50 kV/cm, from 20 kV/cm to 40 kV/cm, from 25 kV/cm to 150 kV/cm, from 25 kV/cm to 100 kV/cm, from 25 kV/cm to 75 kV/cm, or from 25 kV/cm to 50 kV/cm, is applied. In some embodiments, a pulse amplitude of at least 10 kV/cm is applied. In some embodiments, a pulse amplitude of at least 20 kV/cm is applied. In some embodiments, a pulse amplitude of at least 25 kV/cm is applied. In some embodiments, a pulse amplitude of at least 30 kV/cm is applied. In some embodiments, a pulse amplitude of about 35 kV/cm is applied.

In some embodiments, pulses are applied for a duration of about 50 ns to about 900 ns, e.g., from 50 ns to 750 ns, from 50 ns to 500 ns, from 50 ns to 250 ns, from 50 ns to 150 ns, from about 100 ns to 500 ns, from 100 ns to 250 ns, or from 100 ns to 200 ns. In some embodiments, pulses are applied for a duration of about 50 ns, about 100 ns, about 150 ns, about 200 ns, about 250 ns, about 300 ns, about 350 ns, about 400 ns, about 450 ns, about 500 ns, about 600 ns, about 700 ns, about 800 ns, or about 900 ns. In some embodiments, pulses are applied for a duration of about 100 ns.

In some embodiments, the total number of pulses that is applied is at least about 30 pulses, e.g., 50-100 pulses, or about 200-300 pulses, about 400 pulses, 500 pulses, 600 pulses, 700 pulses, 800 pulses, 900 pulses, 1000 pulses or more. In some embodiments, from about 100 pulses to about 1000 pulses are applied, e.g., from about 200 pulses to about 900 pulses, from about 300 pulses to about 800 pulses, from about 300 pulses to about 700 pulses, from about 300 pulses to about 600 pulses, from about 400 pulses to about 900 pulses, from about 400 pulses to about 800 pulses, or from about 500 pulses to about 1000 pulses. In some embodiments, about 50 pulses are applied. In some embodiments, about 100 pulses are applied. In some embodiments, for a given treatment session, the pulses are applied in one or more sets, e.g., in two sets or three sets. In some embodiments, in between sets of pulses, the electrode assembly that is used for delivering the pulses can be repositioned, e.g., in order to effectively treat the site of the growth or tumor.

In some embodiments, the pulses are applied at a rate of up to 6 pulses per second (pps), e.g., from 1 pps to 6 pps, from 2 pps to 6 pps, or from 3 pps to 6 pps. In some embodiments, the pulses are applied at a rate of less than 6 pps. In some embodiments, the pulses are applied at a rate of no more than 5 pps, e.g., up to 4 pps or up to 3 pps. In some embodiments, the pulses are applied at a rate of about 1 pps to about 5 pps, e.g., from about 2 pps to about 5 pps, from about 2 pps to about 4 pps, or from about 3 pps to about 5 pps. In some embodiments, the pulses are applied at a rate of about 3 pps.

### Number of Treatments

In some embodiments, treatment with sub-microsecond pulses is efficacious for the treatment of an HPV-associated growth (e.g., benign growth, pre-cancerous growth, or HPV-associated cancer) if the HPV-associated growth is reduced in size or volume by at least 10%, 20%, 30%, 40%, 50%, 60%, 70%, 80%, or 90% as compared to the size or volume of the growth prior to the treatment. In some embodiments, treatment results in a reduction of the HPV-associated growth to a size or volume that below the level of detection. In some embodiments, a single treatment session with sub-microsecond high voltage pulses is efficacious in treating the HPV-associated growth. In some embodiments, two, three, four, five, or more treatment sessions are administered for treating the HPV-associated growth.

In some embodiments, treatment with sub-microsecond pulses is efficacious for inhibiting the recurrence of an HPV-associated growth (e.g., benign growth, pre-cancerous growth, or HPV-associated cancer) or the metastasis of a cancer if the growth does not recur or if a tumor does not metastasize for a period of at least weeks or months after administration of the treatment. In some embodiments, treatment is efficacious if the growth does not recur or the tumor does not metastasize for at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, or at least 15 weeks after administration of the treatment. In some embodiments, the growth does not recur or the tumor does not metastasize for at least 1 month, at least 2 months, at least 3 months, at least 4 months, at least 5 months, at least 6 months, at least 7 months, at least 8 months, at least 9 months, at least 10 months, at least 11 months, or at least 12 months after administration of the treatment.

In some embodiments, two or more treatment sessions are administered to the subject, e.g., 2, 3, 4, 5, 6, or more treatment sessions. In some embodiments, two or more treatment sessions are administered to a subject in order to treat a plurality of growths on or in the subject. In some embodiments, the two or more treatment sessions are administered at approximately the same time (e.g., on the same day or within 1, 2, 3, 4, 5, 6, or 7 days of each other).

In some embodiments, a second treatment session is administered at least days, weeks, or months after administration of the first treatment session. In some embodiments, a second treatment session is administered at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, or at least 15 weeks after administration of the first treatment session. In some embodiments, a second treatment session is administered at least 1 month, at least 2 months, at least 3 months, at least 4 months, at least 5 months, at least 6 months, at least 7 months, at least 8 months, at least 9 months, at least 10 months, at least 11 months, or at least 12 months after administration of the first treatment session.

### IV. Devices and Computer Systems

In another aspect, devices, machine-readable non-transitory media, and computer systems for controlling a high voltage pulse generator and delivering treatment of sub-microsecond pulses of an electric field are provided. In some embodiments, the devices, machine-readable non-transitory media, and computer systems are used in the treatment of an HPV-associated growth (e.g., a benign growth, a pre-cancerous growth, or a cancerous growth) or for inhibiting the recurrence of an HPV-associated growth or a metastasis of a cancer (e.g., an HPV-associated cancer).

In some embodiments, a device for delivering treatment of sub-microsecond pulses of an electric field is provided. In some embodiments, the device comprises:
a pulse generator configured to generate electric pulses;
an electrode assembly configured to deliver the electric pulses to a site of an abnormal growth in or on a subject, wherein the pulse generator and the electrode assembly are configured to apply a plurality of sub-microsecond pulses of an electric field with an intensity of 1 kV/cm or greater to the site of the abnormal growth; and
a computer processor operatively connected with a machine-readable non-transitory medium, the medium embodying information indicative of instructions for causing the computer processor to perform operations comprising:
   controlling the pulse generator to generate a number of pulses in a treatment session, each pulse having a pulse duration and a pulse amplitude calculated to deliver the pulses at a rate of energy deposition of up to 14 watts/cc.

The pulse generator may be any pulse generator that is capable of generating sub-microsecond pulses at a voltage of 1 kV/cm or greater. Examples of such pulse generators are disclosed, for example, in U.S. Pat. Nos. 6,326,177, 7,767,433, and 8,115,343, incorporated by reference herein.

The electrode assembly may be any device that can deliver the electrical pulses to the subject (e.g., to a site of an abnormal growth in or on a subject, such as an HPV-associated growth or a tumor site). In some embodiments, the electrode assembly comprises at least one delivery (e.g., active) electrode. In some embodiments, the electrode assembly further comprises at least one ground electrode. In some embodiments, the electrode assembly comprises one or more plate electrodes, needle electrodes, or semicircular electrodes.

In some embodiments, a computer system for controlling a high voltage pulse generator is provided. In some embodiments, the system comprises:
a processor; and
a memory operatively coupled with the processor, the processor executing instructions from the memory comprising:
   program code for controlling a pulse generator to generate a number of pulses in a treatment session, each pulse having a pulse duration and a pulse amplitude calculated to deliver the pulses at a rate of energy deposition of up to 14 watts/cc to a site of an abnormal growth in or on a subject through an electrode assembly coupled to the pulse generator.

In some embodiments, a machine-readable non-transitory medium embodying information indicative of instructions for causing a device comprising a pulse generator to perform operations for delivering treatment of sub-microsecond pulses of an electric field is provided. In some embodiments, the operations comprise:
controlling the pulse generator to generate a number of pulses in a treatment session, each pulse having a pulse duration and a pulse amplitude calculated to deliver the pulses at a rate of energy deposition of up to 14 watts/cc to a site of an abnormal growth in or on a subject through an electrode assembly coupled to the pulse generator.

For the devices, computer systems, and machine-readable non-transitory media disclosed herein, in some embodiments, the operations and/or program code comprise controlling the pulse generator to generate a pulse duration, a pulse amplitude, and a total number of pulses and to deliver the pulses at a rate of energy deposition as disclosed in Section III above.

In some embodiments, the operations and/or program code comprise controlling the pulse generator to deliver the pulses at a rate of energy deposition that is up to about 14 watts/cc, e.g., up to about 12 watts/cc, up to about 10 watts/cc, or up to about 8 watts/cc. In some embodiments, the operations and/or program code comprise controlling the pulse generator to deliver the pulses at a rate of energy deposition that is, for example, from about 3 watts/cc to about 6 watts/cc.

In some embodiments, the operations and/or program code comprise controlling the pulse generator to deliver the pulses at a rate of energy deposition that is less than 14 watts/cc, e.g., less than 12 watts/cc, e.g., less than 11 watts/cc, less than 10 watts/cc, less than 9 watts/cc, or less than 8 watts/cc. In some embodiments, the operations and/or program code comprise controlling the pulse generator to deliver the pulses at a rate of energy deposition that is above 0 watts/cc and to less than 14 watts/cc.

In some embodiments, the operations and/or program code comprise controlling the pulse generator to deliver the pulses at a rate of energy deposition that is, for example, about 0.1 watts/cc, about 0.2 watts/cc, about 0.5 watts/cc, about 1 watt/cc, about 1.5 watts/cc, about 2 watts/cc, about 3 watts/cc, about 4 watts/cc, about 5 watts/cc, about 6 watts/cc, about 7 watts/cc, about 8 watts/cc, about 9 watts/cc, about 10 watts/cc, about 11 watts/cc, about 12 watts/cc or about 13 watts/cc.

In some embodiments, the operations and/or program code comprise controlling the pulse generator to generate pulses having a pulse length of about 50 to 900 nanoseconds, e.g., e.g., from 50 ns to 750 ns, from 50 ns to 500 ns, from 50 ns to 250 ns, from 50 ns to 150 ns, from about 100 ns to 500 ns, from 100 ns to 250 ns, or from 100 ns to 200 ns. In some embodiments, the operations and/or program code comprise controlling the pulse generator to generate pulses having a pulse length of about 100 ns.

In some embodiments, the operations and/or program code comprise controlling the pulse generator to generate pulses having a pulse amplitude of about 10 to 200 kV/cm, e.g., from 10 kV/cm to 150 kV/cm, from 10 kV/cm to 100 kV/cm, from 10 kV/cm to 50 kV/cm, from 10 kV/cm to 30 kV/cm, from 15 kV/cm to 45 kV/cm, from 15 kV/cm to 35 kV/cm, from 20 kV/cm to 200 kV/cm, from 20 kV/cm to 150 kV/cm, from about 20 kV/cm to 100 kV/cm, from 20 kV/cm to 50 kV/cm, from 20 kV/cm to 40 kV/cm, from 25 kV/cm to 150 kV/cm, from 25 kV/cm to 100 kV/cm, from 25 kV/cm to 75 kV/cm, or from 25 kV/cm to 50 kV/cm, is applied. In some embodiments, the operations and/or program code comprise controlling the pulse generator to generate pulses having a pulse amplitude of at least 10 kV/cm, e.g., at least 20 kV/cm, at least 25 kV/cm, or at least 30 kV/cm.

In some embodiments, the operations and/or program code comprise controlling the pulse generator to generate at least about 30 pulses, e.g., at least about 50 pulses, 100 pulses, 200 pulses, 300 pulses, 400 pulses, 500 pulses, 600 pulses, 700 pulses, 800 pulses, 900 pulses, 1000 pulses or more. In some embodiments, the operations and/or program code comprise controlling the pulse generator to generate from about 30 pulses to about 1000 pulses, e.g., from about 30 pulses to about 900 pulses, from about 50 pulses to about 800 pulses, from about 100 pulses to about 700 pulses, from about 300 pulses to about 600 pulses, from about 400 pulses to about 900 pulses, from about 400 pulses to about 800 pulses, or from about 500 pulses to about 1000 pulses.

### V. Examples

The following examples are offered to illustrate, but not to limit, the claimed invention.

### Example 1. Nano-Pulse Stimulation Induces Immunogenic Cell Death in Human Papillomavirus-Transformed Tumors and Initiates an Adaptive Immune Response

### Abstract

Nano-Pulse Stimulation (NPS) is a non-thermal pulsed electric field modality that has been shown to have therapeutic effects in treating cancers such as melanomas. As described herein, NPS treatment was applied to the human papillomavirus (HPV)-transformed C3.43 mouse tumor model and was shown to be effective at eliminating primary tumors through the induction of immunogenic cell death while subsequently increasing the number of tumor-infiltrating lymphocytes within the tumor microenvironment. In vitro NPS treatment of C3.43 cells resulted in a 2-fold increase in activated caspase 3/7 along with the translocation of phosphatidylserine (PS) to the outer leaflet of the plasma membrane, suggesting programmed cell death activity. Tumor-bearing mice receiving standard NPS treatment showed an initial decrease in tumor volume followed by clearing of tumors in most mice, significantly increasing overall survival. Intra-tumor analysis of mice that were not able to clear tumors showed an inverse correlation between the number of tumor infiltrating lymphocytes and the size of the tumor. Approximately half of the mice that cleared tumors with this treatment dosage were protected against tumor re-challenge on the opposite flank. Selective depletion of CD8⁺ T cells eliminated this protection, suggesting that NPS treatment induces an adaptive immune response. Eliciting this immune response was dependent on the rate of NPS, as an increased rate of application did not result in tumor re-challenge protection. These results suggests that optimized NPS activates immunogenic cell death leading to the generation of CD8⁺ T cells that recognize tumor antigen(s) associated with the C3.43 tumor model. These data demonstrate that NPS can be utilized to not only ablate primary tumors, but also to induce an anti-tumor response driven by effector CD8⁺ T cells capable of eliminating secondary tumors while also protecting individuals from disease recurrence.

NPS is a non-thermal tumor treatment modality that uses ultra-short electric pulses to induce immunogenic cell death in treated tissues. NPS has been applied to non-viral tumor types and has been shown to induce immunogenic cell death that then leads to necrosis and slow regression over a period of weeks. See, Beebe et al., FASEB J., 2003, 17:1493-1495; Nuccitelli et al., Biochem Biophys Res Commun, 2006, 343:351-360; Nuccitelli et al., Int J Cancer, 2009, 125:438-445; and Nuccitelli et al., Int J Cancer, 2010, 127:1727-1736. It has been found that after NPS treatment, cells of the innate immune system are recruited to the treated tumor and phagocytose tumor cells. Within 3 weeks, CD8⁺ cytotoxic T cells are generated that target the tumor cells. Nuccitelli et al., Pigment Cell Melanoma Res, 2012, 25:618-629; Chen et al., Eur J Cancer, 2014, 50:2705-2713; and Nuccitelli et al., PLoS One, 2015, 10(7):e0134364.

In order to determine if NPS could be effective at eliminating HPV-transformed tumors, in vitro treatment effects of NPS on the HPV16-transformed murine tumor cell line C3.43 with respect to caspase activation and PS translocation to the outer leaflet of the plasma membrane, as well as in vivo treatment effects on established subdermal C3.43-tumors in immunocompetent mice, were examined.

### Materials and Methods

Specific pathogen-free female C57BL/6 (B6) mice, 6 to 8 weeks old, were purchased from Taconic Farms. Tumor challenge studies were performed using the C3.43 cell line (Smith et al., Clinical Cancer Research, 2009, 15:6167-6176), an in vivo passaged derivative of the C3 HPV16-transformed B6 murine tumor cell line (Feltkamp et al., Eur J Immunol, 1993, 23:2242-2249). C3.43 cells have retained expression of the HPV16 E6 and E7 (by reverse transcription-PCR and Western blot), express similar levels of MHC class I molecules on the surface compared with the parental C3 line (as measured by flow cytometry), and respond to prophylactic vaccination with HPV16 E7-containing vaccines in vivo. C3.43 cells tested negative for Mycoplasma contamination (MycoAlert Mycoplasma Detection kit, Lonza, Walkersville, Md.). Cells used for tumor challenge were cultured for 10-11 days from seed stocks in Iscove's modified Dulbecco's medium supplemented with 10% fetal bovine serum before in vivo challenge. All procedures were performed in accordance with institutional guidelines and approved by the University of Southern California Institutional Animal Care and Use Committee. The following phenotyping antibodies were purchased from BioLegend (San Diego, Calif.): CD3 FITC (clone 145-2C11), CD4 PE-Cy5.5 (clone GK 1.5), CD8a PE-Cy7 (clone 53-6.7), CD45 APC-Cy7 (clone 30-F11), rat IgG2a FITC, rat IgG1 PE-Cy5, rat IgG2b PE-Cy7, and rat IgG2b APC-Cy7.

All treatments were applied with a ns pulse generator (Transient Plasma Systems, Torrance Calif.) that was tuned to deliver a relatively square pulse into a 200 ohm load using magnetic compression technology. Pulses were applied at either 3 or 6 pulses per second (pps). The typical pulse rise time was 25 ns and a typical pulse delivered 65-80 A of current at 30 kV/cm and 97-120 A at 50 kV/cm. 30 kV/cm was most commonly used, delivering approximately 0.1 J of energy into the tumor 3 times per second, delivering 0.3 watts. The volume of the treatment zone was 0.085 cc, so the rate of energy deposition was 3.5 watts/cc at 3 pps and 30 kV/cm and this rate increased to 14 watts/cc at 6 pps and 50 kV/cm.

Tumor Challenge and NPS Treatment:

Groups of 10 to 15 eight-week-old female C57BL/6 mice were challenged subcutaneously in the right flank with 5×10⁵ C3.43 tumor cells suspended in 100 μl HBSS. Ten days after tumor cell injection, once tumors had grown to a mean diameter of 3-5 mm, groups receiving NPS were treated. For example, 600 pulses 100 ns long and either 30 or 50 kV/cm in amplitude were applied at either 3 or 6 pps using an electrode that sandwiched the tumor between two flat cylindrical polished stainless-steel plates 6 mm wide with a spacing of 3 mm between the two plates. The pulses were applied, 300 pulses at a time, and the electrode was repositioned over the tumor between applications to ensure coverage of the entire tumor. Throughout the duration of the experiments tumor growth and overall survival was assessed. Tumor size was measured two to three times per week via caliper and volume (mm³) was calculated based on L×W×H. Mice were euthanized when tumor volume exceeded 1,500 mm³ or if ulceration occurred. In experiments examining efficacy of therapeutic vaccination with HPV16-Venezuelan Equine Encephalitis replicon particle (VRP) (Cassetti et al., Vaccine, 2004, 22:529-527), mice were vaccinated with 1.0×10⁷ infectious units of VRP i.m. in 50 μL PBS on days 14 and 21 post tumor challenge. For tumor re-challenge experiments, mice were given 5×10⁵ C3.43 cells subcutaneously on the opposite side of the primary tumor.

Tumor-Infiltrating Lymphocytes (TIL) Isolation and Flow Cytometry Phenotyping:

Tumors were isolated from individual mice and processed into single cell suspensions using a mouse tumor dissociation kit with the GentleMACS system (Miltenyi, Auburn, Calif.) according to manufacturer's instructions. Cell suspension was passed through a 70 mm nylon strainer to generate a single cell population and TIL separated from tumor cells and debris via a Lympholyte-M gradient (Cedarlane, Burlington, N.C.). Isolated TILs were incubated with 1:200 dilution of Zombie Aqua (Biolegend, San Diego, Calif.) to stain for dead cells, washed twice with PBS, incubated with Fc block (Biolegend) for 30 minutes on ice, and then stained for surface antigens indicated by flow panel for 1 hour at 4° C. After washing, cells were fixed with FluoroFix buffer containing 1% paraformaldehyde (Biolegend), washed, and collected via flow cytometry. A minimum of 20,000 CD45⁺ events were acquired on the BD FACSCanto II. Flow data were analyzed utilizing FlowJo software (ver. 10.3). Populations were first gated on viable cells using a Zombie Aqua live/dead indicator dye, and CD45⁺ to indicate lymphocyte population. The following sub-gate markers were used for specific populations: CD3⁺CD4⁺ (CD4 T cells), CD3⁺CD8⁺ (CD8 T cells).

CD4 and CD8 T Cell Ablation Assays:

Groups of 10-12 C57Bl/6 mice were challenged with 1.0×10⁵ C3.43 tumor cells and NPS treated as described above. Post NPS treatment, mice were randomized into three groups. Mice that cleared tumors through NPS treatment were then subjected to selective CD4 or CD8 depletion 28 days post initial tumor challenge and 3 days before tumor re-challenge as indicated. Depletion was carried out by 3 consecutive daily IP doses of 500 μg α-CD4 (clone GK1.5) or α-CD8 (clone 2.43) antibodies followed by maintenance dosing every 3^{rd} consecutive day for the duration of the experiment. Depletion was confirmed by weekly flow cytometry analysis of circulating cells collected through retro orbital bleeds—CD3⁺CD4⁺ (CD4 T cells), CD3⁺CD8⁺ (CD8 T cells). All groups were re-challenged at day 31, including a fourth group of age-matched naïve mice to control for tumor take. Tumor growth and survival was monitored as described above.

Annexin V/7-AAD Apoptosis Detection Assay:

The PE Annexin V Apoptosis Detection Kit I (BD Biosciences) was used to assay the percentage of cells undergoing the stages of apoptotic cell death. Cells were treated with NPS (as described above) and then incubated at 37° C. with 5% CO₂. Cells were harvested at 1, 3 and 24 hours post treatment. After harvesting, cells were washed twice with 1×PBS (wash: suspend in 100 μl buffer; centrifuged at 1200 rpm for 5 minutes at 4° C.) followed by resuspension in 100 μl of Annexin V/7-AAD (7-Aminoactinomycin D) staining cocktail (1 μl PE Annexin V and 1 μl 7-AAD in 100 μl 1× Annexin Binding Buffer). Cells were protected from light and incubated for 15 minutes at room temperature. After incubation, 100 μl binding buffer was added to each sample and gently mixed. Stained cells were analyzed on a Beckman CytoFLEX flow cytometer. Cells were gated based upon Annexin V binding (PE Annexin V: Ex 488/Em 578) and cell viability (7-AAD: Ex 488/Em 647). Gated cells were binned into four populations based upon stage of cell death: live viable cells (PE Annexin V−/7-AAD−); early apoptotic (still viable) (PE Annexin V+/-AAD−); late stage apoptotic/necrotic (non-viable) (PE Annexin V+/17-AAD+), very late stage cell death (non-viable) (PE Annexin V−/7-AAD+). Binned populations were expressed as % of total cells.

Activated Caspase Assay:

Activation of combined caspase-3 and caspase-7 was assessed using the Caspase-Glo® 3/7 Assay (Promega). Following NPS treatments, 1.5×10⁴ C3.43 cells were plated in triplicates within a 96-well assay plate containing pre-equilibrated media and incubated for 3 hours at 37° C., and 5% CO₂. Caspase-Glo reagent was added to each well at a ratio of 1:1 with cell culture media. This reagent contains pro-luminescent caspase-3/7 substrate, which contains the tetrapeptide sequence, DEVD. This substrate is cleaved to release amino-luciferin, a substrate of luciferase used in the production of light. Cell lysis results, followed by caspase cleavage of the substrate and generation of luminescence. Samples were incubated for an additional 30 minutes at room temperature, protected from light, and gently agitated. Sample luminescence was then measured using the Molecular Devices SpectraMax i3 plate reader. Caspase activation was normalized to untreated samples by dividing the raw luminescence units (RLUs) of pulsed samples by the RLU value of untreated controls.

IFN-Gamma Enzyme Linked Immunospot (ELISpot) Assay:

96-well ELISpot plates (Millipore Multiscreen HTS IP) were coated with 5 μg/ml IFNγ capture Ab (Clone AN18, BD Biosciences) in sterile PBS overnight at 4° C. Plates were washed twice with sterile PBS. Complete RMPI medium was then used to block plates for 2 hours at 37° C. Splenocytes isolated from treated mice were plated in triplicate at 5×10⁵ cells per well in regular medium or medium containing a final concentration of either 2 μg/ml of HPV16 E749-57 peptide (Feltkamp et al., Eur J Immunol, 1993, 23:2242-2249), 2 μg/ml of ampitope peptide Pakhomov et al., Bioelectromagnetics, 2007, 28:655-663), or 1 μg/ml of Concanavalin A (Sigma Chemical Co.). After 20 hours of incubation at 37° C., plates were washed six times with 0.05% PBST and were incubated with 1 μg/ml of biotinylated IFNγ antibody (Clone R4-6A2, BD Biosciences) in PBS containing 0.5% BSA for 2 hours at room temperature. Plates were washed six times with 0.05% PBST and wells were subsequently incubated with 100 μl of 1:4000 diluted streptavidin-horseradish peroxidase (Sigma Chemical Co.) for 1 hour at room temperature. Spots were developed using an AEC (3-amino-9-ethyl-carbazole) (Sigma Chemical Co.) substrate for 5 minutes and reactions were quenched with deionized water. A Zeiss KS ELISPOT microscope was used to determine the number of spots per well. HPV16 E749-57 specific T cells were quantified after subtraction of background spots from medium control wells.

### Results

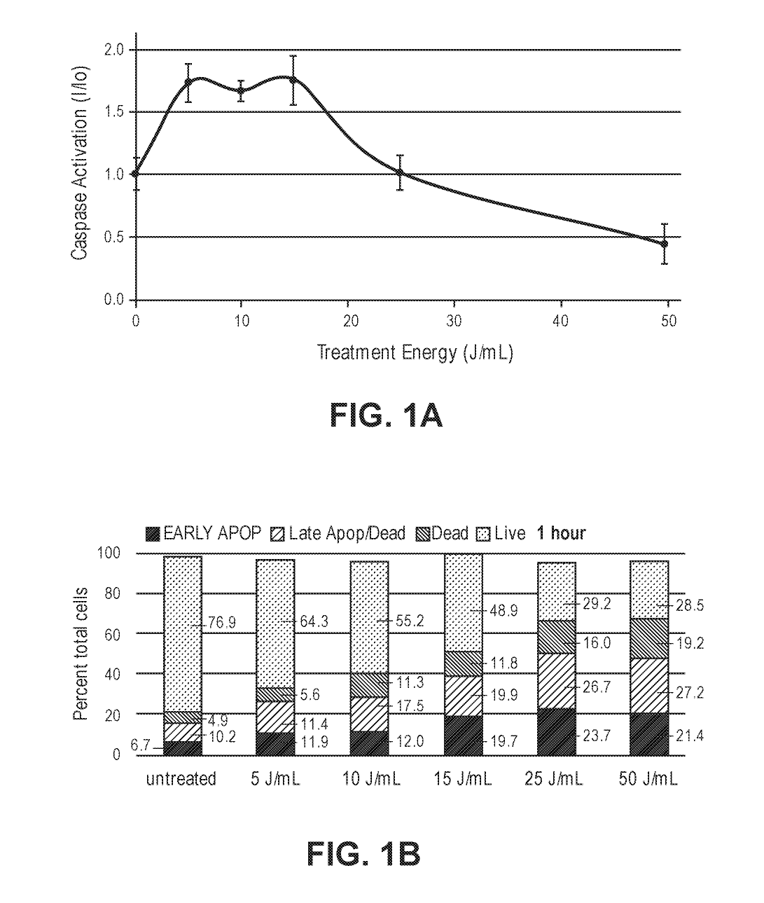

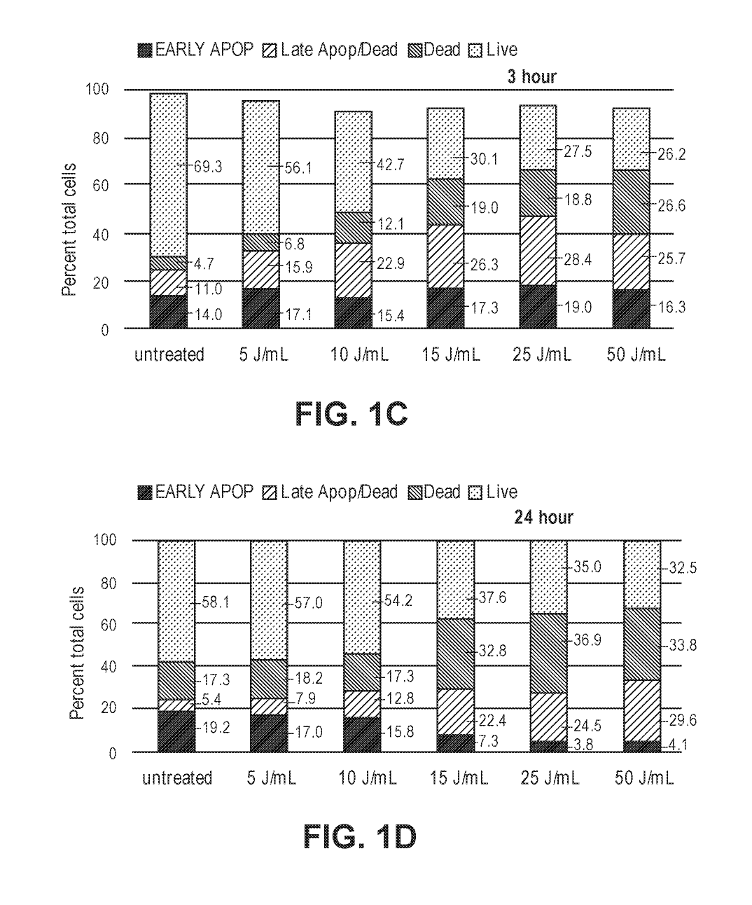

C3.43 cells were treated with NPS by placing the cells in an electroporation cuvette and pulsing them with a range of pulse numbers using 15 kV/cm, 100 ns-long pulses at 2 pps. The amount of activated caspase 3/7 was measured 1 hour, 3 hours, and 24 hours after NPS treatment. As shown in FIG. 1A, a 2-fold increase was found at 5 J/ml, which was very similar to the optimal energy for stimulating caspase activation in three other cell lines that have been studied (Nuccitelli et al., Journal for Immunotherapy of Cancer, 2017, 5:32-45). As a second measure of apoptosis, NPS-treated C3.43 cells were analyzed using the PE Annexin V apoptosis detection kit performed by flow cytometry (FIGS. 1B-1D). Within one hour, about 50% of the cells treated with 15-50 J/ml were in early or late apoptosis compared to only 17% of untreated cells. This percentage did not change at 3 hours, but the number of dead cells increased at both 3 hours and 24 hours. This is the expected progression from apoptosis to necrosis following NPS treatment. These results demonstrate that NPS triggers immunogenic cell death in C3.43 cells.

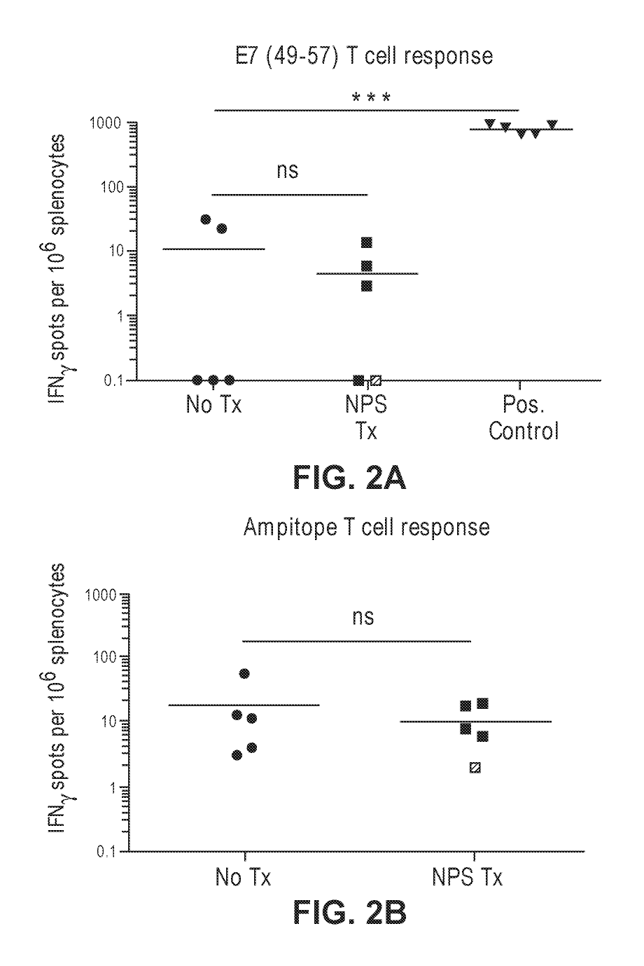

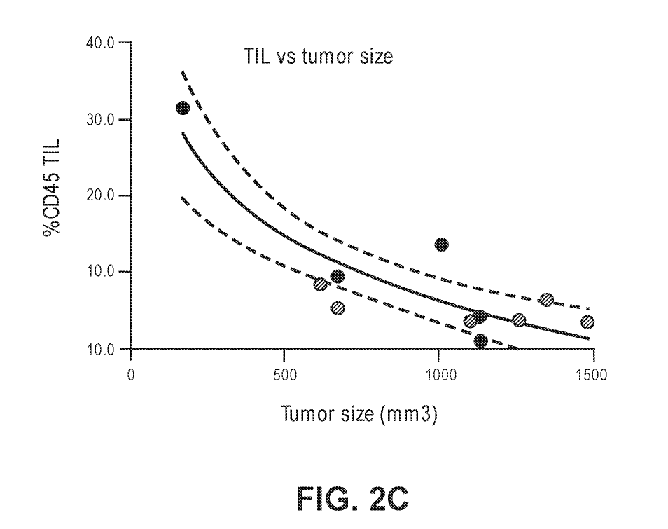

Splenocytes isolated from both tumor-bearing naïve mice and mice that had undergone NPS-treatment of primary tumors were incubated with peptides associated with anti-HPV16 responses as well as a secondary epitope from the ampicillin resistance gene (ampitope) that is known to be expressed on the C3.43 cell line (van Hall et al., Cancer Res, 19982, 58:3087-3093). Groups of five mice were analyzed for either HPV16 E7 specific T cells or for recognition of the ampitope, found on the C3.43 model, with mice vaccinated using a VRP-based vaccine serving as a positive control for the E7 target. As shown in FIG. 2A, no significant differences in number of spots were seen between non-treated and NPS-treated mice (***=p<0.001, one-way ANOVA followed by Tukey's multiple comparison test, comparisons to the naïve group shown). In FIG. 2A, black squares indicate mice that had cleared tumors post NPS treatment while the striped square was a mouse with a recurrent tumor. As shown in FIG. 2B, no significant differences in number of spots were seen between non-treated and NPS treated mice for the ampitope target (unpaired students t-test). These results indicate that NPS treatment generates effector CD8⁺ T cells that respond to unidentified antigens in the C3.43 model.

The percentage of CD45⁺ cells were plotted against tumor volumes for mice from ELISpot and survival experiments. As shown in FIG. 2C, nonlinear regression analysis of tumor volume (x-axis) plotted against percentage of CD45⁺ TILs detected via flow cytometry (y-axis) showed that there was an inverse correlation between the size of the tumor and the percentage of CD45⁺ TILs found within isolates (p<0.0003 with the null hypothesis that slope=0, R²=0.79). This suggests that in mice that have smaller tumors post NPS treatment, there are a greater number of lymphocytes present, which may help explain the reduced tumor volumes and slow rate of growth.

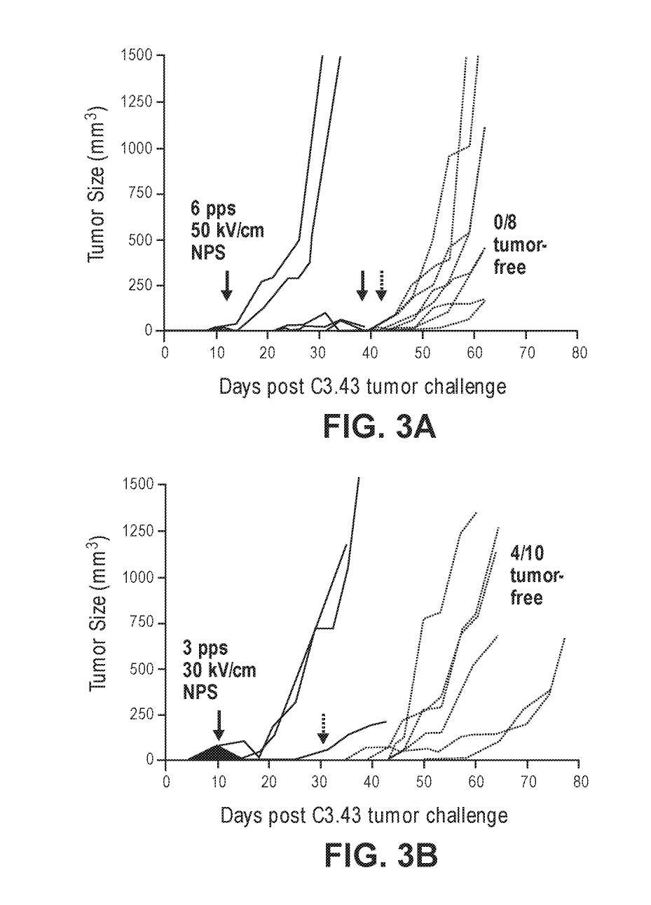

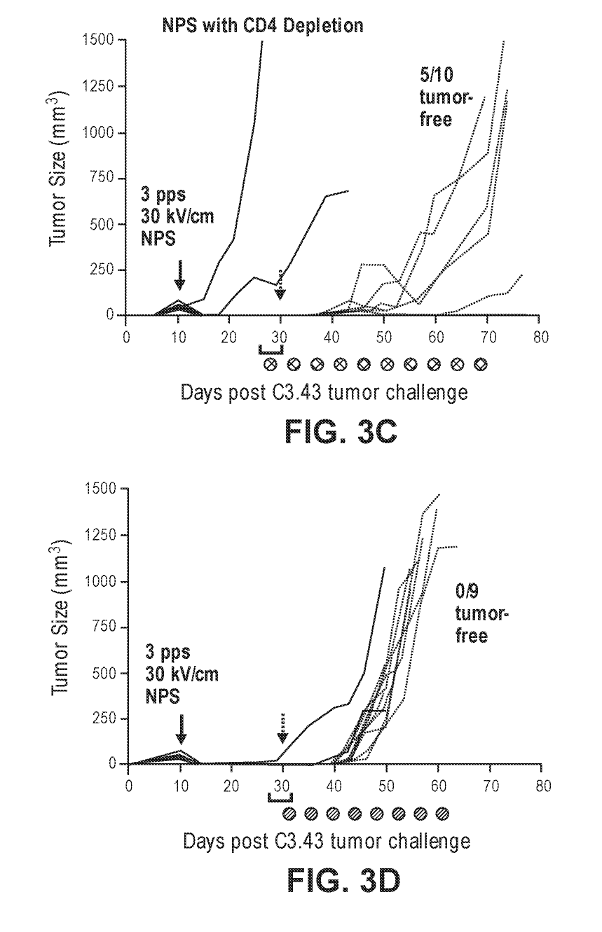

It was discovered that a rate of NPS application in vivo makes a difference for triggering an immune response. For example, when NPS is applied at a rapid rate and high field strength (6 pps, 50 kV/cm, 120 A) resulting in 14 watt/cc, 80% of the tumors were ablated and this is similar to the ablation rate observed when NPS was applied more moderately (compare solid lines in growth curves of FIGS. 3A and 3B). However, the rejection of re-challenge tumors is significantly different following these two NPS treatments. Upon re-challenge 3 weeks after the rapid-rate (e.g., 6 pps, 50 kV/cm, 120 A) NPS treatment, all of the re-challenge tumors grew, albeit at a wide range of growth rates (FIG. 3A, dotted lines). In contrast, when NPS was applied at a more moderate rate (e.g., 3 pps, 30 kV/cm, 69 A) resulting in 3 watts/ml, 40% of the mice that were re-challenged with C3.43 cells remained tumor-free (FIG. 3B, dotted lines).

Mice bearing approximately 30 mm³ tumors were randomized into four groups of 10. The tumors in three of these groups were treated with NPS and the fourth group became the naïve control. Several days prior to tumor re-challenge, two of the NPS-treated groups were selectively either CD4- or CD8-depleted by intra-peritoneal administration of three doses of anti-CD4 or anti-CD8 antibody. Depletion of specific populations of CD4 and CD8 was verified by flow cytometry from blood collected via retro-orbital bleeds and verified on a bi-weekly basis during maintenance dosing of depletion antibodies. Tumor re-challenge was carried out on all four groups.

FIG. 3C shows that selective depletion of CD4 with administration of an αCD4 mAb (hatched circles) had a similar rejection rate to slowly applied NPS-treated mice. FIG. 3D shows that selective depletion of CD8 with the administration of an αCD8 mAb (striped circles) eliminated the protection against tumor re-challenge, suggesting the presence of an adaptive immune response against primary tumors. The dotted arrows in FIGS. 3C-3D indicate the day of tumor re-challenge. The growth curves for both the primary NPS-treated tumors (solid lines) and the re-challenge tumors (dotted lines) show that CD8-depleted mice (FIG. 3D) had the tumor growth and survival profile of the naïve group while CD4-depleted mice (FIG. 3C) showed significantly improved survival and significantly smaller tumors at Day 60. Thus, CD8 depletion in mice with NPS-treated primary tumors resulted in loss of protection against tumor re-challenge. These results suggest that selective CD8 depletion in mice eliminates the protective effects of treating primary tumors with NPS and that NPS treatment of primary tumors induces an adaptive immune response against neo-antigens of C3.43 tumors that have yet to be defined.

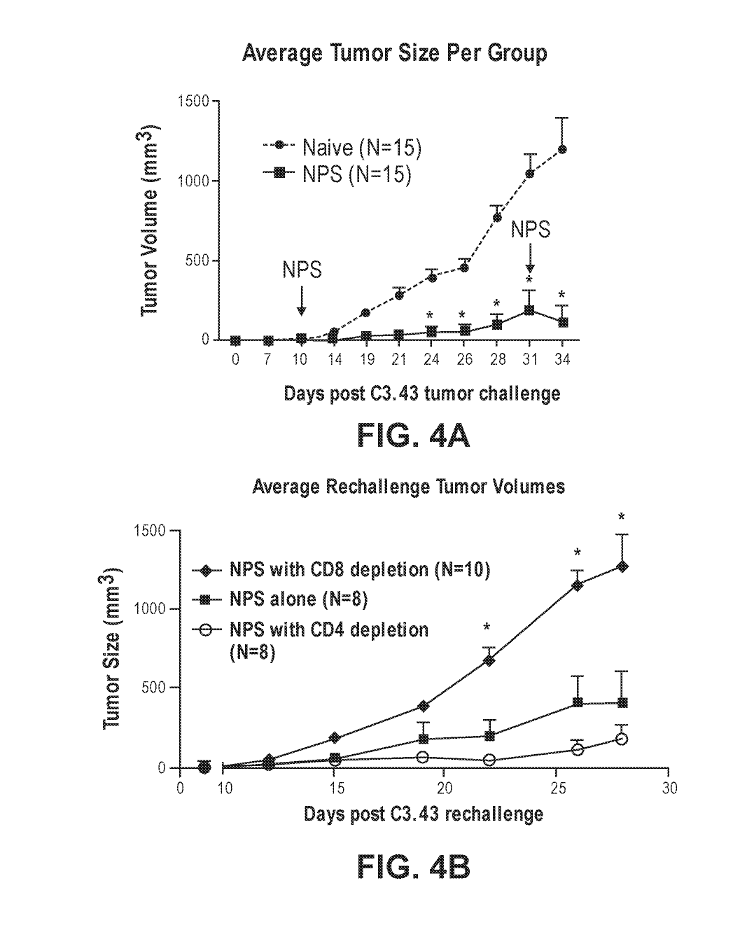

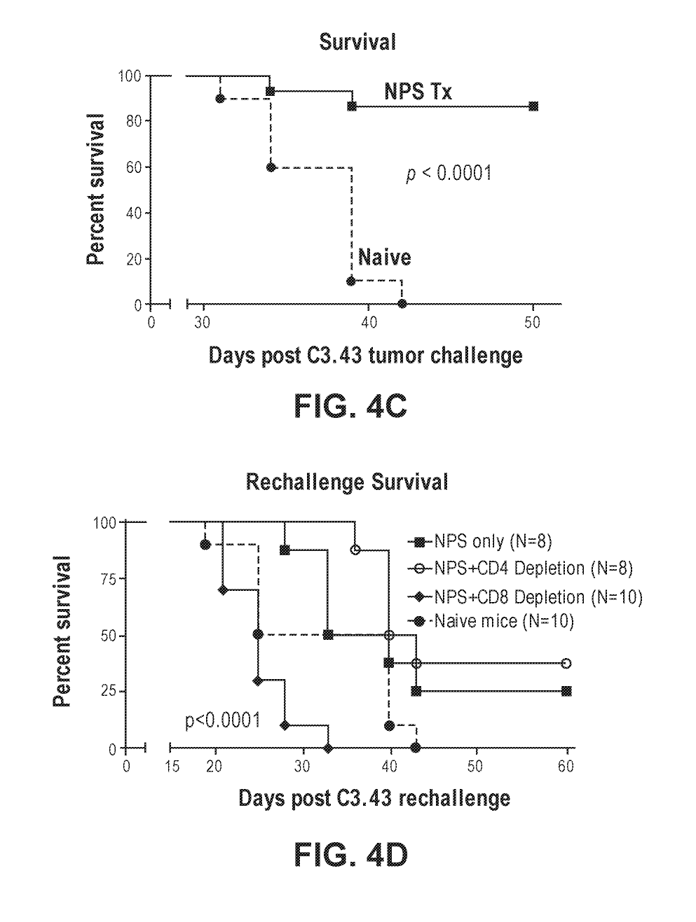

Groups of 15 mice were subcutaneously challenged with C3.43 tumors. 10 days post tumor challenge, mice were given NPS (6 pps, 50 kV/cm) treatment at the tumor site. Mice with recurring tumors received a second treatment on day 31. As shown in FIG. 4A, the mean tumor volume (±SEM) of each group showed a significant decrease in NPS-treated mice beginning at day 24 (*p<0.05, unpaired students t-test at each time point). Nearly all primary tumors were ablated (FIG. 4A), resulting in a significant increase in overall survival for these mice (FIG. 4C; p<0.0001, Mantel-Cox Log Rank test). Groups of mice (n=8-10) that had cleared tumors in initial NPS treatment (3 pps, 30 kV/cm) were re-challenged subcutaneously with C3.43 tumors on the contralateral side. Prior to challenge, indicated groups were dosed with anti-CD4 or anti-CD8 antibodies three days prior to tumor challenge followed by maintenance dosing every third day. As shown in FIG. 4B, a significant difference in average tumor volume was seen starting at day 23 in CD8-depleted mice when compared to NPS treated mice (*p<0.05, One-way ANOVA followed by Dunnets multiple comparison test to the NPS treated groups. Re-challenge tumors grew at nearly the same rate as primary tumors in age-matched naïve mice when CD8 cells were depleted. As shown in FIG. 4D, survival analysis of mice receiving a second tumor challenge showed significant improvements in median survival of CD4-depleted (41.5 days) or NPS-treated mice without depletion (36.5 days) while CD8-depleted mice showed similar median survival to naïve challenged mice (25 and 32.5 days, respectively) (p<0.0001, Mantel-Cox Log-rank test). These results demonstrate that NPS treatment of primary C3.43 tumors results in significant tumor reduction, increase in survival, and a high level of tumor clearance.

As described above, it is discovered by the inventors of the present disclosure that the initiation of an immune response is dependent on the rate of NPS application. Specifically, it was found that when NPS is applied at the level of 14 watts/cc, there seemed to be a very weak adaptive immune response as mice were not protected from tumor re-challenge, but showed some delay in tumor rechallenge growth (FIG. 3A). If there had been an absence of an adaptive response after treatment, tumor growth similar to the CD8-depleted group (FIG. 3D) would have been expected. A possible explanation for this observation is that at an NPS rate of 14 watts/cc, hyperthermia is generated within the tumor that then leads to necrosis rather than immunogenic cell death. Based on the heat capacity of water being 4.2 J/° C., the 2.3 J/s applied to the tumor may raise the temperature at a rate of 0.55° C./s assuming that all of the energy was trapped within the skin in the pinch electrode. Over the treatment time of 100 seconds, this could heat the tumor to 55° C., which is in the range to cause hyperthermia that leads to tumor necrosis (Nikfarjam et al., J Gastrointest Surg, 2005, 9:410-417). While treatment at this power-level would still eliminate the primary tumor, necrosis does not generally lead to an immune response as observed with immunogenic cell death. This dependence of the immune response on the rate of NPS application is quite interesting and relevant for the successful implementation of NPS treatment of virus-associated tumors in the future.

In summary, NPS can be used to eliminate HPV-transformed tumors effectively with a single treatment lasting several minutes. This modality of treatment has the potential to not only ablate primary tumors in patients, but also generate an immune response against HPV-tumors, ultimately resulting in the protection of individuals from tumor recurrence or elimination of metastatic events.

It is understood that the various embodiments described herein are by way of example only, and are not intended to limit the scope of the invention. For example, many of the materials and structures described herein may be substituted with other materials and structures without deviating from the spirit of the invention. It should be apparent that individual features of one embodiment or example may be combined with one or more features of another embodiment or features from a plurality of embodiments. As will be appreciated by those skilled in the art, the methods of the present disclosure may be embodied, at least in part, in software and carried out in a computer system or other data processing system. Therefore, in some exemplary embodiments hardware may be used in combination with software instructions to implement the present disclosure. The present invention as claimed may therefore include variations from the particular examples and embodiments described herein, as will be apparent to one of skill in the art. All publications mentioned herein are incorporated herein by reference to disclose and describe the methods and/or materials in connection with which the publications are cited.

What is claimed is:

1. A method of treating a subject having a human papillomavirus (HPV)-associated growth, the method comprising applying a treatment of sub-microsecond pulses of an electric field with an intensity of 1 kV/cm or greater to the subject at a site of the HPV-associated growth, wherein the pulses are applied at a rate of energy deposition of up to about 14 watts/cc;
thereby treating the subject having the HPV-associated growth.

2. The method of claim 1, wherein the HPV-associated growth is a benign growth.

3. The method of claim 2, wherein the benign growth is a wart, a sinonasal papilloma, or a laryngeal papilloma.

4. The method of claim 1, wherein the HPV-associated growth is recurrent respiratory papillomatosis.

5. The method of claim 1, wherein the HPV-associated growth is a pre-cancerous growth or an HPV-associated cancer.

6. The method of claim 1, wherein the pulses are applied at the rate of energy deposition configured to stimulate an immune response to the HPV-associated growth or metastasis.

7. The method of claim 5, wherein the HPV-associated cancer is a HPV-16 associated cancer or a HPV-18 associated cancer.

8. The method of claim 7, wherein the HPV-associated cancer is cervical cancer, vulvar cancer, vaginal cancer, penile cancer, anal cancer, rectal cancer, or oropharyngeal cancer.

9. The method of claim 1, wherein the subject is a human.

10. The method of claim 1, wherein the pulses have a pulse amplitude of about 10 to 200 kV/cm.

11. The method of claim 10, wherein the pulses have a pulse amplitude of about 30 kV/cm.

12. The method of claim 1, wherein the pulses have a pulse duration of about 50 to 900 nanoseconds.

13. The method of claim 12, wherein each pulse has a pulse duration of about 100 nanoseconds.

14. The method of claim 1, wherein the treatment comprises applying the pulses at a rate of up to 6 pulses per second.

15. A method of inhibiting the recurrence of an HPV-associated growth or a metastasis of a cancer in a subject, the method comprising applying a treatment of sub-microsecond pulses of an electric field with an intensity of 1 kV/cm or greater to a site of the HPV-associated growth or metastasis in or on the subject, wherein the treatment comprises applying the pulses at a rate of energy deposition that is less than 14 watts/cc.

16. The method of claim 15, wherein the treatment comprises applying the pulses at a rate of energy deposition that is less than 10 watts/cc.

17. The method of claim 15, wherein the treatment comprises applying the pulses at a rate of energy deposition of about 3 watts/cc.

18. The method of claim 15, wherein the treatment comprises applying the pulses at a rate of up to 5 pulses per second.

19. The method of claim 15, wherein the treatment inhibits the recurrence of an HPV-associated growth and the HPV-associated growth is a benign growth.

20. The method of claim 15, wherein the treatment inhibits the metastasis of a virus associated cancer.

21. The method of claim 20, wherein the virus-associated cancer is an HPV-associated cancer.

22. The method of claim 21, wherein the HPV-associated cancer is a HPV-16 associated cancer.

23. The method of claim 21, wherein the HPV-associated cancer is cervical cancer, vulvar cancer, vaginal cancer, penile cancer, anal cancer, rectal cancer, or oropharyngeal cancer.

24. A device for delivering treatment of sub-microsecond pulses of an electric field, comprising:
a pulse generator configured to generate electric pulses;
an electrode assembly configured to deliver the electric pulses to a site of an abnormal growth in or on a subject, wherein the pulse generator and the electrode assembly are configured to apply a plurality of sub-microsecond pulses of an electric field with an intensity of 1 kV/cm or greater to the site of the abnormal growth; and
a processor operatively connected with a machine-readable non-transitory medium, the medium embodying information indicative of instructions for causing the processor to perform operations comprising:
controlling the pulse generator to generate a number of pulses in a treatment session, each pulse having a pulse duration and a pulse amplitude calculated to deliver the pulses at a rate of energy deposition of up to 14 watts/cc.

25. The device of claim 24, wherein the operations comprise controlling the pulse generator to deliver the pulses at a rate of energy deposition that is less than 10 watts/cc.

26. The device of claim 24, wherein the operations comprise controlling the pulse generator to deliver the pulses at a rate of energy deposition of about 2 to 4 watts/cc.

27. The device of claim 24, the device comprising a user interface and configured to allow a user to modify one or more parameters of pulses or other parameters of operation of the pulse generator.

28. The device of claim 24, wherein the abnormal growth is an HPV-associated growth.

29. A machine-readable non-transitory medium embodying information indicative of instructions for causing a device comprising a pulse generator to perform operations for delivering treatment of sub-microsecond pulses of an electric field, the operations comprising:
controlling the pulse generator to generate a number of pulses in a treatment session, each pulse having a pulse duration and a pulse amplitude calculated to deliver the pulses at a rate of energy deposition of up to 14 watts/cc to a site of an abnormal growth in or on a subject through an electrode assembly coupled to the pulse generator.

30. A computer system for controlling a high voltage pulse generator, the system comprising:
a processor; and
a memory operatively coupled with the processor, the processor executing instructions from the memory comprising:
program code for controlling a pulse generator to generate a number of pulses in a treatment session, each pulse having a pulse duration and a pulse amplitude calculated to deliver the pulses at a rate of energy deposition of up to 14 watts/cc to a site of an abnormal growth in or on a subject through an electrode assembly coupled to the pulse generator.
